(19) United States
(12) Patent Application Publication
(10) Pub. No.: US 2022/0076820 A1
(43) Pub. Date: Mar. 10, 2022

(54) METHODS AND APPARATUS FOR BIOMETRIC DATA

(71) Applicant: InBody Co., Ltd., Seoul (KR)

(72) Inventor: Ki Chul Cha, Seoul (KR)

(21) Appl. No.: 17/017,203

(22) Filed: Sep. 10, 2020

(51) Int. Cl. G16H 40/67; G06T 11/20

(52) U.S. Cl. CPC G16H 40/67; G06T 11/206

(57) ABSTRACT

Disclosed is a method and system for processing biometric data. The disclosed biometric data processing method includes measuring biometric data of an examinee; determining a standard range in a graph corresponding to the biometric data; and providing the examinee with visualized information by overlaying the standard range and the biometric data on the graph. The graph includes one axis based on age and another axis based on the biometric data, and the standard range is a range that is determined according to the age based on pre-stored reference data.

### TECHNICAL FIELD

The following description relates to a method and apparatus for processing biometric data processing method.

### BACKGROUND ART

Anthropometry refers to an important indicator for evaluating health and, in terms of health management, it is important to evaluate a health and nutritional status of an individual by comparing changes in anthropometric values and normal changes.

Meanwhile, biometric data includes various types of information measured from a body of an examinee. A standard range may vary based on biometric data, which makes it difficult to interpret biometric data measured from the examinee.

### DISCLOSURE OF INVENTION

### Technical Solution

A biometric data processing method according to an example embodiment includes measuring biometric data of an examinee; determining a standard range in a graph corresponding to the biometric data; and providing the examinee with visualized information by overlaying the standard range and the biometric data on the graph. The graph includes one axis based on age and another axis based on the biometric data.

The biometric data may include Whole Body ECW Ratio (ECW/TBW) data about a female, the standard range may be a range that is determined according to the age based on pre-stored reference data, at the age of 20, the upper limit of the standard range may have a value of 0.382 or more and 0.383 or less, and the lower limit of the standard range may have a value of 0.370 or more and 0.372 or less, at the age of 30, the upper limit of the standard range may have a value of 0.383 or more and 0.384 or less, and the lower limit of the standard range may have a value of 0.372 or more and 0.373 or less, at the age of 40, the upper limit of the standard range may have a value of 0.384 or more and 0.386 or less, and the lower limit of the standard range may have a value of 0.373 or more and 0.375 or less, at the age of 50, the upper limit of the standard range may have a value of 0.386 or more and 0.389 or less, the lower limit of the standard range may have a value of 0.375 or more and 0.378 or less, and at the age of 60, the upper limit of the standard range may have a value of 0.390 or more and 0.394 or less, and the lower limit of the standard range may have a value of 0.379 or more and 0.382 or less.

The biometric data may include Visceral Fat Area (VFA) data about a female, the standard range may be a range that is determined according to the age based on pre-stored reference data, at the age of 20, the upper limit of the standard range may have a value of 112.0 or more and 124.3 or less, and the lower limit of the standard range may have a value of 39.3 or more and 42.5 or less, at the age of 30, the upper limit of the standard range may have a value of 120.9 or more and 147.7 or less, and the lower limit of the standard range may have a value of 42.8 or more and 50.0 or less, at the age of 40, the upper limit of the standard range may have a value of 142.8 or more and 159.7 or less, and the lower limit of the standard range may have a value of 49.8 or more and 58.4 or less, at the age of 50, the upper limit of the standard range may have a value of 154.8 or more and 175.4 or less, and the lower limit of the standard range may have a value of 58.3 or more and 69.2 or less, and at the age of 60, the upper limit of the standard range may have a value of 172.3 or more and 186.7 or less, and the lower limit of the standard range may have a value of 66.9 or more and 80.4 or less.

The biometric data may include Body Mass Index (BMI) data about a female, the standard range may be a range that is determined according to the age based on pre-stored reference data, at the age of 20, the upper limit of the standard range may have a value of 28.9 or more and 30.9 or less, and the lower limit of the standard range may have a value of 20.7 or more and 21.4 or less, at the age of 30, the upper limit of the standard range may have a value of 31.0 or more and 31.9 or less, and the lower limit of the standard range may have a value of 21.5 or more and 22.1 or less, at the age of 40, the upper limit of the standard range may have a value of 32.0 or more and 32.5 or less, and the lower limit of the standard range may have a value of 22.2 or more and 22.6 or less, at the age of 50, the upper limit of the standard range may have a value of 32.2 or more and 32.4 or less, and the lower limit of the standard range may have a value of 22.4 or more and 22.6 or less, and at the age of 60, the upper limit of the standard range may have a value of 32.0 or more and 32.5 or less, and the lower limit of the standard range may have a value of 22.3 or more and 22.5 or less.

The biometric data may include Whole Body Phase Angle data about a female, the standard range may be a range that is determined according to the age based on pre-stored reference data, at the age of 20, the upper limit of the standard range may have a value of 6.20 or more and 6.28 or less, and the lower limit of the standard range may have a value of 5.09 or more and 5.13 or less, at the age of 30, the upper limit of the standard range may have a value of 6.15 or more and 6.21 or less, and the lower limit of the standard range may have a value of 5.05 or more and 5.11 or less, at the age of 40, the upper limit of the standard range may have a value of 5.94 or more and 6.15 or less, and the lower limit of the standard range may have a value of 4.88 or more and 5.08 or less, at the age of 50, the upper limit of the standard range may have a value of 5.63 or more and 5.93 or less, and the lower limit of the standard range may have a value of 4.60 or more and 4.86 or less, and at the age of 60, the upper limit of the standard range may have a value of 5.19 or more and 5.58 or less, and the lower limit of the standard range may have a value of 4.28 or more and 4.57 or less.

The biometric data may include Waist Hip Ratio (WHR) data about a female, the standard range may be a range that is determined according to the age based on pre-stored reference data, at the age of 20, the upper limit of the standard range may have a value of 0.97 or more and 0.99 or less, and the lower limit of the standard range may have a value of 0.83 or more and 0.84 or less, at the age of 30, the upper limit of the standard range may have a value of 0.99 or more and 1.01 or less, and the lower limit of the standard range may have a value of 0.84 or more and 0.85 or less, at the age of 40, the upper limit of the standard range may have a value of 1.01 or more and 1.02 or less, and the lower limit of the standard range may have a value of 0.85 or more and 0.86 or less, at the age of 50, the upper limit of the standard range may have a value of 1.02 or more and 1.03 or less, and the lower limit of the standard range may have a value of 0.86 or more and 0.87 or less, and at the age of 60, the upper limit of the standard range may have a value of 1.03 or more and 1.03 or less, and the lower limit of the standard range may have a value of 0.87 or more and 0.87 or less.

The biometric data may include Fat Free Mass Index (FFMI) data about a female, the standard range may be a range that is determined according to the age based on pre-stored reference data, at the age of 20, the upper limit of the standard range may have a value of 18.5 or more and 19.1 or less, and the lower limit of the standard range may have a value of 15.5 or more and 15.8 or less, at the age of 30, the upper limit of the standard range may have a value of 19.1 or more and 19.3 or less, and the lower limit of the standard range may have a value of 15.8 or more and 16.1 or less, at the age of 40, the upper limit of the standard range may have a value of 19.1 or more and 19.3 or less, and the lower limit of the standard range may have a value of 16.0 or more and 16.1 or less, at the age of 50, the upper limit of the standard range may have a value of 18.5 or more and 19.0 or less, and the lower limit of the standard range may have a value of 15.6 or more and 15.9 or less, and at the age of 60, the upper limit of the standard range may have a value of 18.1 or more and 18.4 or less, and the lower limit of the standard range may have a value of 15.2 or more and 15.5 or less.

The biometric data may include Percent Body Fat (PBF) data, the standard range may be a range that is determined according to the age based on pre-stored reference data, at the age of 20, the upper limit of the standard range may have a value of 38.6 or more and 40.8 or less, and the lower limit of the standard range may have a value of 22.0 or more and 23.0 or less, at the age of 30, the upper limit of the standard range may have a value of 40.9 or more and 42.1 or less, and the lower limit of the standard range may have a value of 23.3 or more and 24.4 or less, at the age of 40, the upper limit of the standard range may have a value of 42.1 or more and 43.5 or less, and the lower limit of the standard range may have a value of 24.6 or more and 26.3 or less, at the age of 50, the upper limit of the standard range may have a value of 43.5 or more and 44.7 or less, and the lower limit of the standard range may have a value of 26.3 or more and 28.2 or less, and at the age of 60, the upper limit of the standard range may have a value of 44.9 or more and 45.8 or less, and the lower limit of the standard range may have a value of 28.1 or more and 29.7 or less.

The biometric data may include Weight data about a female, the standard range may be a range that is determined according to the age based on pre-stored reference data, at the age of 20, the upper limit of the standard range may have a value of 78.6 or more and 84.1 or less, and the lower limit of the standard range may have a value of 55.2 or more and 57.3 or less, at the age of 30, the upper limit of the standard range may have a value of 84.3 or more and 86.8 or less, and the lower limit of the standard range may have a value of 57.3 or more and 59.1 or less, at the age of 40, the upper limit of the standard range may have a value of 86.9 or more and 88.0 or less, and the lower limit of the standard range may have a value of 59.2 or more and 60.1 or less, at the age of 50, the upper limit of the standard range may have a value of 86.7 or more and 88.0 or less, and the lower limit of the standard range may have a value of 59.5 or more and 60.1 or less, and at the age of 60, the upper limit of the standard range may have a value of 84.5 or more and 86.8 or less, and the lower limit of the standard range may have a value of 58.2 or more and 59.3 or less.

### BRIEF DESCRIPTION OF DRAWINGS

FIG. 1 illustrates a biometric data processing apparatus according to an example embodiment.

FIG. 2 illustrates a result screen output according to an example embodiment.

FIGS. 3A to 10D illustrate examples of a graph displayed on a result screen according to an example embodiment.

FIG. 11 is a flowchart illustrating a biometric data processing method according to an example embodiment.

### DETAILED DESCRIPTION FOR CARRYING OUT THE INVENTION

The following structural or functional descriptions of example embodiments described herein are merely intended for the purpose of describing the example embodiments described herein and may be implemented in various forms. Here, the example embodiments are not construed as limited to specific implementation and should be understood to include all changes, equivalents, and replacements within the idea and the technical scope of the disclosure.

Although terms of “first,” “second,” and the like are used to explain various components, the components are not limited to such terms. These terms are used only to distinguish one component from another component. For example, a first component may be referred to as a second component and, likewise, a second component may be referred to as a first component.

Also, when it is mentioned that one component is “connected” to another component, it may be understood that the one component is directly connected or accessed to another component or that still other component is interposed between the two components.

As used herein, the singular forms are intended to include the plural forms as well, unless the context clearly indicates otherwise. It will be further understood that the terms “comprises” or “comprising,” when used in this specification, specify the presence of stated features, integers, steps, operations, elements, components or a combination thereof, but do not preclude the presence or addition of one or more other features, integers, steps, operations, elements, components, or combinations thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

Hereinafter, example embodiments are described in detail with reference to the accompanying drawings. The following specific structural and functional descriptions are merely provided to explain the example embodiments and the scope of the example embodiments should not be construed as limited to the example embodiments set forth herein. Those skilled in the art may make various alterations and modifications from the description. Also, like reference numerals in the drawings refer to like elements and known functions and structures are omitted.

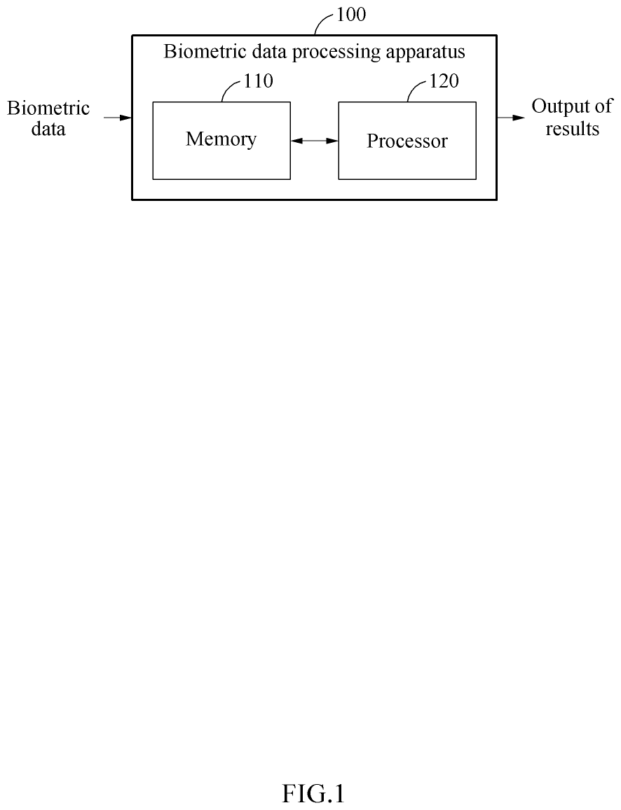

FIG. 1 illustrates a biometric data processing apparatus according to an example embodiment.

Referring to FIG. 1, a biometric data processing apparatus 100 includes a memory 110 and a processor 120.

The biometric data processing apparatus 100 measures biometric data of an examinee. The biometric data includes data about Whole Body ECW Ratio (ECW/TBW), Visceral Fat Area (VFA, cm²), Body Mass Index (BMI, kg/m²), Whole Body Phase Angle_50 kHz (PhA, °), Waist Hip Ratio (WHR), Fat Free Mass Index (FFMI, kg/m²), Percent Body Fat (PBF, %), Weight (kg), and the like.

The biometric data processing apparatus 100 determines a graph corresponding to biometric data. The graph includes one axis based on age and another axis based on the biometric data. Also, the graph includes a predetermined range that corresponds to age and is based on pre-measured biometric data. Here, the predetermined range may represent the average range of corresponding age. For simplicity of description, the average range used herein may be represented as a standard range.

The biometric data processing apparatus 100 displays the measured biometric data on the graph. The biometric data processing apparatus 100 may display the biometric data on the graph to correspond to age of an examinee. Since visualized information is provided to the examinee by overlaying the measured biometric data of the examinee on the graph including the predetermined range according to the age, the examinee may intuitively recognize the healthy degree of his or her physical condition.

The memory 110 may include a computer-readable instruction. The processor 120 may perform the aforementioned operations in response to execution of the instruction stored in the memory 110 on the processor 120. The memory 110 may be a volatile memory or a non-volatile memory.

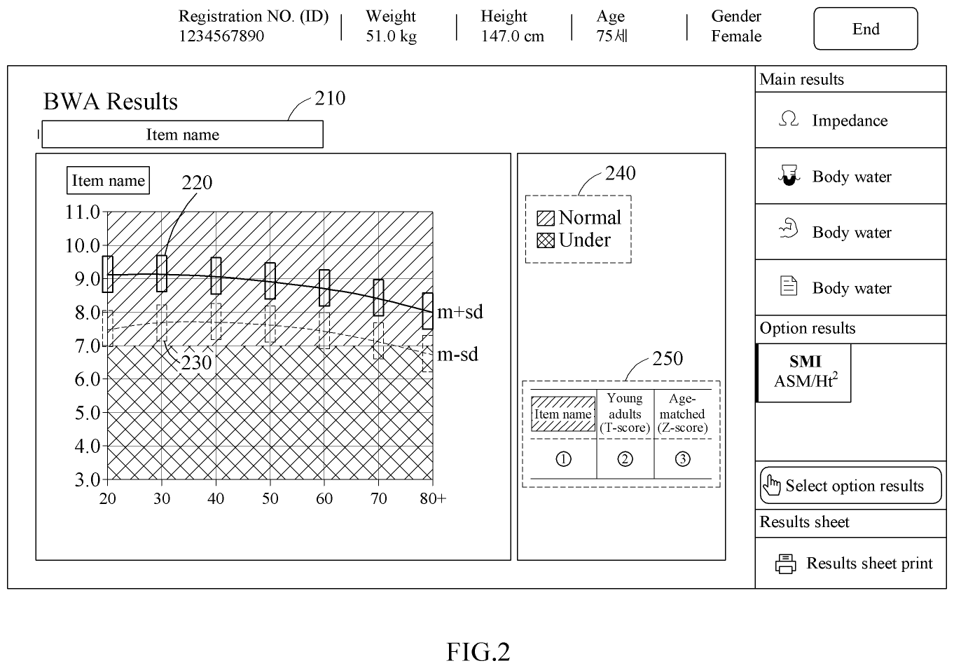

FIG. 2 illustrates a result screen output according to an example embodiment.

The graph of FIG. 2 may correspond to specific biometric data. Such biometric data may be represented as an item name 210. In the graph, a horizontal axis may be based on age and a vertical axis may be based on biometric data. Referring to the graph of FIG. 2, there are two lines and the range defined with the two lines may represent the average range. The average range may be determined based on biometric data pre-measured according to age. Between the two lines, an upper line may represent the upper limit of the average range and a lower line may represent the lower limit of the average range. Referring to FIG. 2, for example, the upper limit of the average range corresponding to 30s may be determined within a first range 220 and the lower limit thereof may be determined within a second range 230. Likewise, the upper limit and the lower limit of the average range may be determined in the same manner for the remaining age groups.

When biometric data of the examinee is included in the average range, that is, when biometric data of the examinee has a value between the upper limit and the lower limit of the average range, the examinee may intuitively recognize that the examinee has the average physical condition at the age of the examinee. Alternatively, when biometric data of the examinee is positioned higher than the average range in the graph, the examinee may intuitively recognize that the examinee has a poor physical condition at the age of the examinee. Alternatively, when biometric data of the examinee is positioned lower than the average range in the graph, the examinee may intuitively recognize that the examinee has a good physical condition at the age of the examinee. On the contrary, based on a type of biometric data, when biometric data of the examinee is positioned higher than the average range in the graph, it may represent that the examinee has a good physical condition at the age of the examinee. When biometric data of the examinee is positioned lower than the average range in the graph, the examinee may intuitively recognize that the examinee has a poor physical condition at the age of the examinee.

For example, the upper limit and the lower limit of the average range may be determined in the following range. In the following example embodiments, “m+sd” may represent the upper limit of the average range and “m−sd” may represent the lower limit of the average range.

TABLE 1
Male

| | | m − sd | m − sd | m + sd | m + sd |
|---|---|---|---|---|---|
| Item name | age | MIN | MAX | MIN | MAX |
| Whole Body ECW | 20 | 0.362 | 0.365 | 0.374 | 0.376 |
| Ratio (ECW/TBW) | 30 | 0.365 | 0.367 | 0.376 | 0.378 |
| | 40 | 0.367 | 0.370 | 0.378 | 0.380 |
| | 50 | 0.370 | 0.373 | 0.381 | 0.385 |
| | 60 | 0.374 | 0.379 | 0.386 | 0.391 |
| Visceral Fat Area | 20 | 18.0 | 30.6 | 77.1 | 99.4 |
| (VFA, cm²) | 30 | 31.6 | 43.6 | 101.0 | 120.1 |
| | 40 | 45.4 | 54.8 | 116.5 | 131.1 |
| | 50 | 53.8 | 65.5 | 127.0 | 141.2 |
| | 60 | 60.5 | 70.0 | 135.5 | 147.6 |
| Body Mass Index | 20 | 21.9 | 23.5 | 29.0 | 31.3 |
| (BMI, kg/m²) | 30 | 23.6 | 24.5 | 31.6 | 32.7 |
| | 40 | 24.6 | 25.1 | 32.8 | 33.2 |
| | 50 | 24.7 | 25.1 | 32.6 | 33.2 |
| | 60 | 24.2 | 24.8 | 32.2 | 32.8 |
| Whole Body Phase | 20 | 6.14 | 6.30 | 7.38 | 7.63 |
| Angle_50 kHz | 30 | 6.05 | 6.18 | 7.20 | 7.41 |
| (PhA, °) | 40 | 5.83 | 6.05 | 6.98 | 7.18 |
| | 50 | 5.50 | 5.81 | 6.54 | 6.90 |
| | 60 | 4.96 | 5.42 | 6.02 | 6.49 |
| Waist Hip Ratio | 20 | 0.81 | 0.84 | 0.96 | 1.01 |
| (WHR) | 30 | 0.84 | 0.86 | 1.02 | 1.04 |
| | 40 | 0.86 | 0.88 | 1.04 | 1.05 |
| | 50 | 0.88 | 0.88 | 1.05 | 1.06 |
| | 60 | 0.88 | 0.89 | 1.06 | 1.07 |
| Fat Free Mass Index | 20 | 18.9 | 19.4 | 23.2 | 23.6 |
| (FFMI, kg/m²) | 30 | 19.4 | 19.7 | 23.7 | 23.8 |
| | 40 | 19.7 | 19.8 | 23.6 | 23.9 |
| | 50 | 19.2 | 19.7 | 22.8 | 23.6 |
| | 60 | 18.5 | 19.2 | 21.9 | 22.8 |
| Percent Body Fat | 20 | 8.8 | 12.6 | 23.9 | 28.5 |
| (PBF, %) | 30 | 12.9 | 15.1 | 29.2 | 31.0 |
| | 40 | 15.4 | 17.0 | 31.1 | 32.2 |
| | 50 | 17.0 | 18.6 | 32.2 | 33.3 |
| | 60 | 18.7 | 20.3 | 33.6 | 35.0 |
| Weight (kg) | 20 | 68.3 | 73.3 | 94.3 | 101.4 |
| | 30 | 73.5 | 76.6 | 102.4 | 106.1 |
| | 40 | 76.8 | 78.3 | 106.0 | 107.3 |
| | 50 | 76.6 | 78.3 | 104.8 | 106.9 |
| | 60 | 74.4 | 77.0 | 102.1 | 105.3 |

TABLE 2
Female

| | | m − sd | m − sd | m + sd | m + sd |
|---|---|---|---|---|---|
| item name | age | MIN | MAX | MIN | MAX |
| Whole Body ECW | 20 | 0.370 | 0.372 | 0.382 | 0.383 |
| Ratio (ECW/TBW) | 30 | 0.372 | 0.373 | 0.383 | 0.384 |
| | 40 | 0.373 | 0.375 | 0.384 | 0.386 |
| | 50 | 0.375 | 0.378 | 0.386 | 0.389 |
| | 60 | 0.379 | 0.382 | 0.390 | 0.394 |
| Visceral Fat Area | 20 | 39.3 | 42.5 | 112.0 | 124.3 |
| (VFA, cm²) | 30 | 42.8 | 50.0 | 120.9 | 147.7 |
| | 40 | 49.8 | 58.4 | 142.8 | 159.7 |
| | 50 | 58.3 | 69.2 | 154.8 | 175.4 |
| | 60 | 66.9 | 80.4 | 172.3 | 186.7 |
| Body Mass Index | 20 | 20.7 | 21.4 | 28.9 | 30.9 |
| (BMI, kg/m²) | 30 | 21.5 | 22.1 | 31.0 | 31.9 |
| | 40 | 22.2 | 22.6 | 32.0 | 32.5 |
| | 50 | 22.4 | 22.6 | 32.2 | 32.4 |
| | 60 | 22.3 | 22.5 | 32.0 | 32.5 |
| Whole Body Phase | 20 | 5.09 | 5.13 | 6.20 | 6.28 |
| Angle_50 kHz | 30 | 5.05 | 5.11 | 6.15 | 6.21 |
| (PhA, °) | 40 | 4.88 | 5.08 | 5.94 | 6.15 |
| | 50 | 4.60 | 4.86 | 5.63 | 5.93 |
| | 60 | 4.28 | 4.57 | 5.19 | 5.58 |
| Waist Hip Ratio | 20 | 0.83 | 0.84 | 0.97 | 0.99 |
| (WHR) | 30 | 0.84 | 0.85 | 0.99 | 1.01 |
| | 40 | 0.85 | 0.86 | 1.01 | 1.02 |
| | 50 | 0.86 | 0.87 | 1.02 | 1.03 |
| | 60 | 0.87 | 0.87 | 1.03 | 1.03 |
| Fat Free Mass Index | 20 | 15.5 | 15.8 | 18.5 | 19.1 |
| (FFMI, kg/m²) | 30 | 15.8 | 16.1 | 19.1 | 19.3 |
| | 40 | 16.0 | 16.1 | 19.1 | 19.3 |
| | 50 | 15.6 | 15.9 | 18.5 | 19.0 |
| | 60 | 15.2 | 15.5 | 18.1 | 18.4 |
| Percent Body Fat | 20 | 22.0 | 23.0 | 38.6 | 40.8 |
| (PBF, %) | 30 | 23.3 | 24.4 | 40.9 | 42.1 |
| | 40 | 24.6 | 26.3 | 42.1 | 43.5 |
| | 50 | 26.3 | 28.2 | 43.5 | 44.7 |
| | 60 | 28.1 | 29.7 | 44.9 | 45.8 |
| Weight (kg) | 20 | 55.2 | 57.3 | 78.6 | 84.1 |
| | 30 | 57.3 | 59.1 | 84.3 | 86.8 |
| | 40 | 59.2 | 60.1 | 86.9 | 88.0 |
| | 50 | 59.5 | 60.1 | 86.7 | 88.0 |
| | 60 | 58.2 | 59.3 | 84.5 | 86.8 |

Specific numerical values described in Table 1 and Table 2 may have the following meaning. Since a standard range is determined based on data of persons measured with an in-body device instead of being determined based on measurement results of the in-body device for all of the persons in the U.S.A., a difference from the US average may occur. In particular, since an in-body measurement device is widely used by persons in their 20s and 30s and a measurement count tends to decrease according to an increase in age, a relatively great difference may occur at the age with a least number of data. The degree of difference between the US average and the average of persons measured with the in-body device may be quantified as a standard error. Here, the standard error may be calculated according to standard deviation/sqrt (number of data). In this manner, the average of all of the persons in the U.S.A. may be derived based on only the in-body measurement results. Here, ±3 sigma may be set such that a probability of including the US average becomes 99.7% based on the standard error of age with the least number of data.

The aforementioned specific numerical values may vary according to race and country.

In addition to the aforementioned example embodiments, various example embodiments may be applied without limitations, such as determining the upper limit of the average range as “mean+arbitrary number” and determining the lower limit of the average range as “mean−arbitrary number.”

A first item 240 may represent an area distinguished from the average range in the graph. For example, an area corresponding to “Normal” may represent a normal physical condition and an area corresponding to “Under” may represent an insufficient physical condition.

Referring to a second item 250, T-score may represent a score acquired by comparing the mean of data corresponding to 20s in pre-measured biometric data and biometric data of the examinee and Z-score may represent a score acquired by comparing the mean of data corresponding to age of the examinee in the pre-measured biometric data and the biometric data of the examinee.

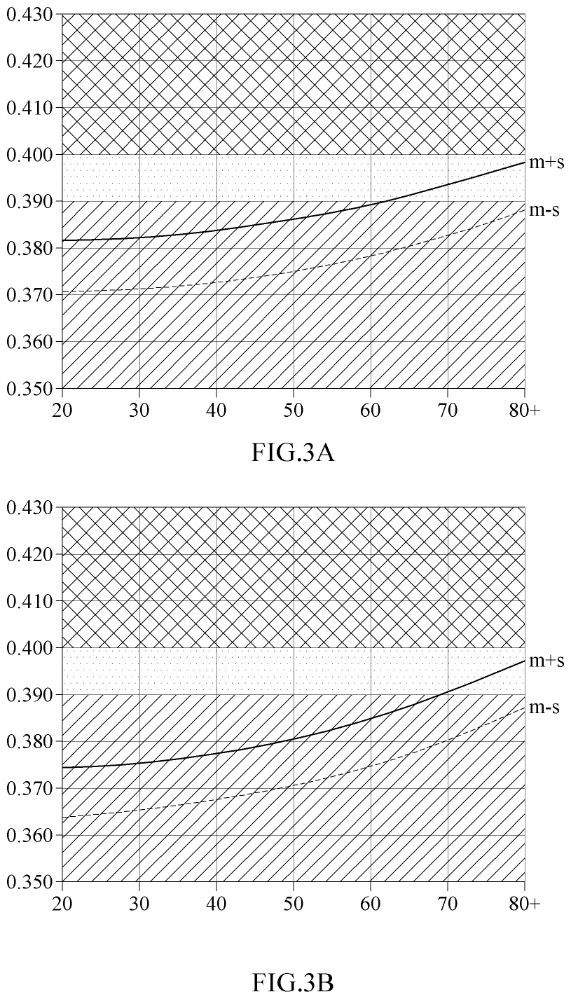

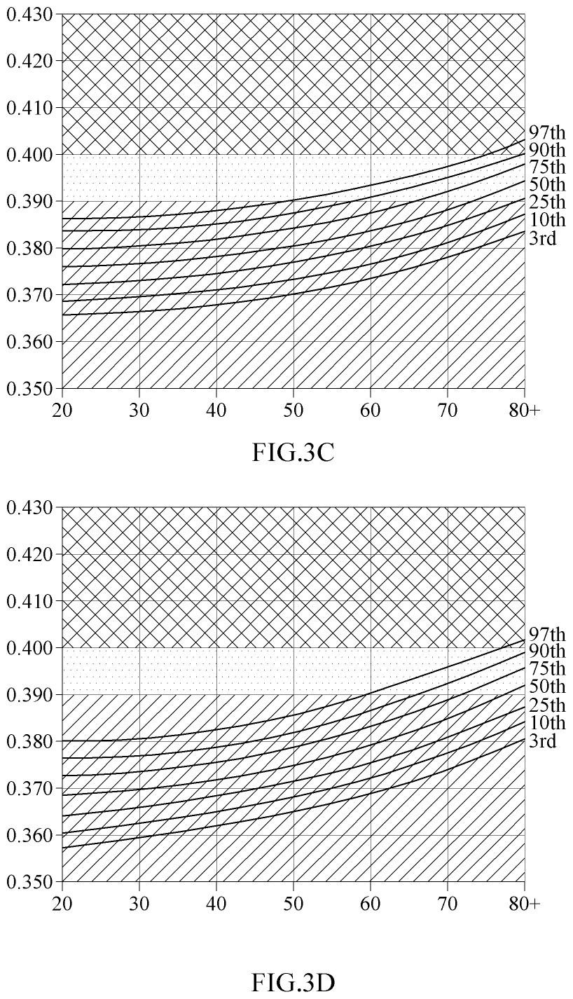

FIGS. 3A to 10D illustrate examples of a graph displayed on a result screen according to an example embodiment.

FIGS. 3A to 3D illustrate graphs corresponding to a Whole Body ECW Ratio (ECW/TBW). For example, FIG. 3A illustrates a graph showing the upper limit and the lower limit of the standard range about a female and FIG. 3B illustrates a graph showing the upper limit and the lower limit of the standard range about a male. Also, FIG. 3C illustrates a percentile graph about a female and FIG. 3D illustrates a percentile graph about a male. Referring to FIGS. 3A to 3D, similar to the first item 240 of FIG. 2, distinguishing areas from the graph may be displayed together. For example, in FIGS. 3A to 3D, areas indicated with grids may correspond to “over,” areas indicated with dots may correspond to “slightly over,” and areas indicated with oblique lines may correspond to “normal.”

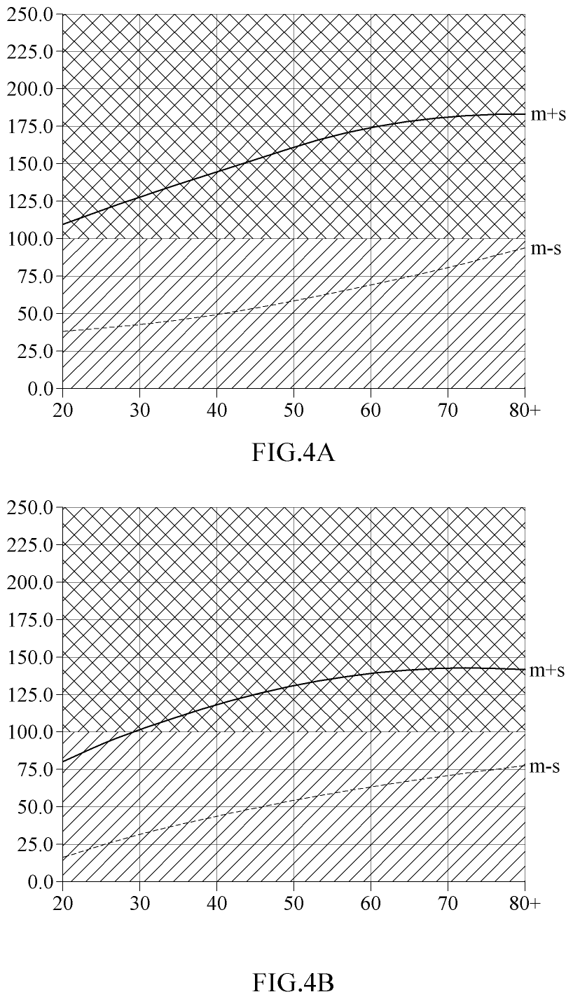

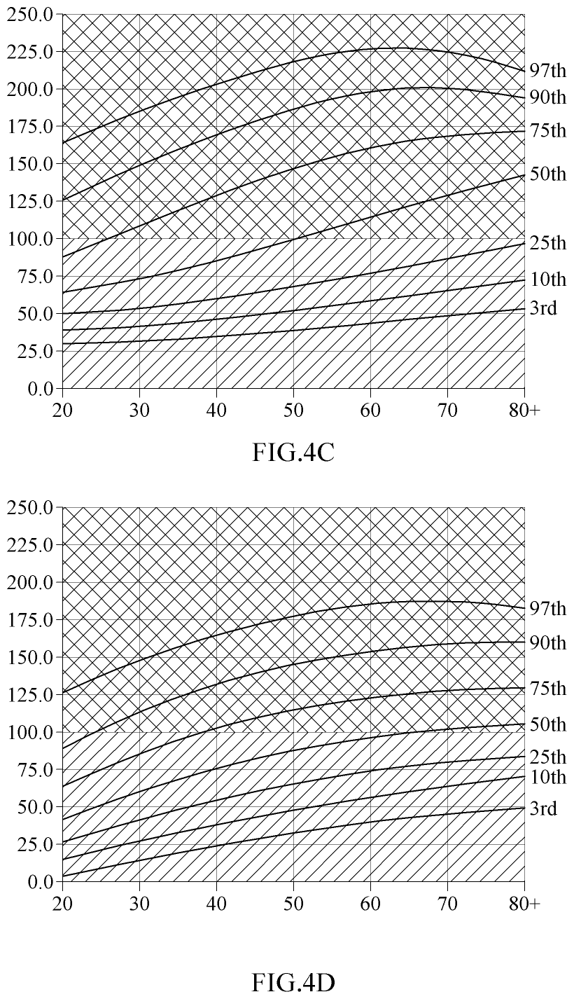

FIGS. 4A to 4D illustrate graphs corresponding to Visceral Fat Area (VFA, cm²). For example, FIG. 4A illustrates a graph showing the upper limit and the lower limit of the standard range about a female and FIG. 4B illustrates a graph showing the upper limit and the lower limit of the standard range about a male. Also, FIG. 4C illustrates a percentile graph about a female and FIG. 4D illustrates a percentile graph about a male. Referring to FIGS. 4A to 4D, similar to the first item 240 of FIG. 2, distinguishing areas from the graph may be displayed together. For example, in FIGS. 4A to 4D, areas indicated with grids may correspond to “over” and areas indicated with oblique lines may correspond to “normal.”

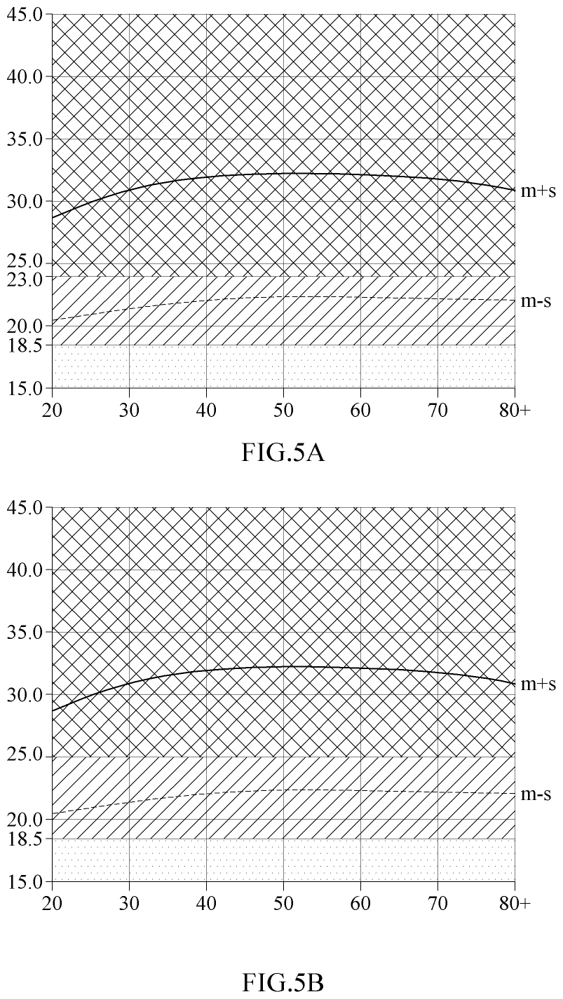

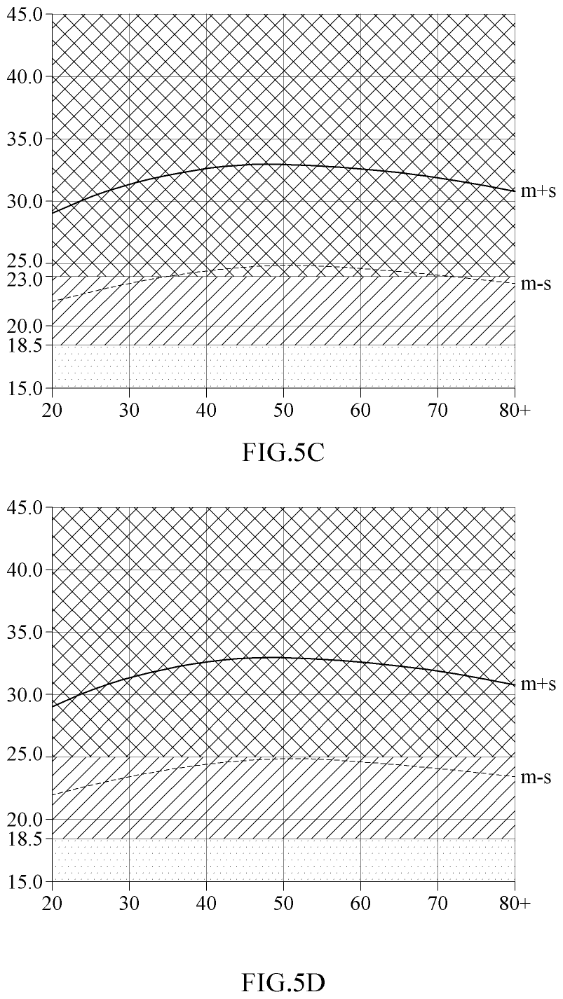

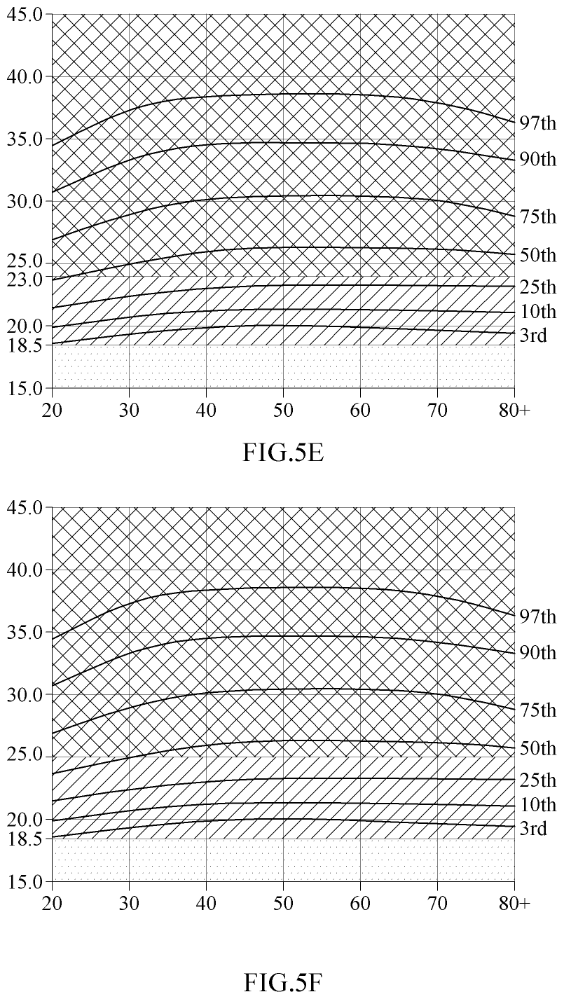

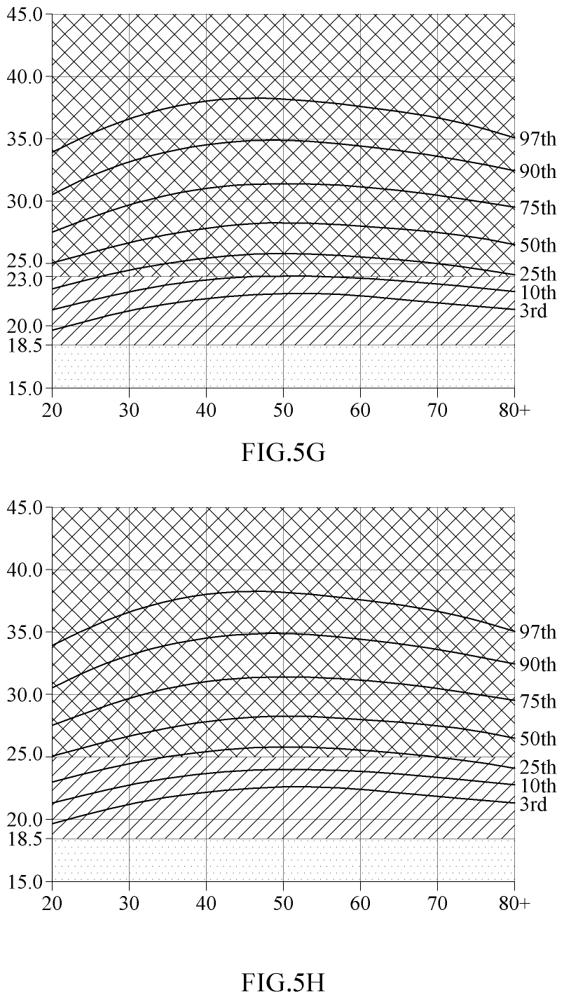

FIGS. 5A to 5H illustrate graphs corresponding to Body Mass Index (BMI, kg/m²). For example, FIGS. 5A and 5B illustrate a case in which cutoff=23.0 and 25.0, respectively, as graphs showing the upper limit and the lower limit of the standard range about a female. FIGS. 5C and 5D illustrate a case in which cutoff=23.0 and 25.0, respectively, as graphs showing the upper limit and the lower limit of the standard range about a male. Also, FIGS. 5E and 5F illustrate a case in which cutoff=23.0 and 25.0, respectively, as graphs showing a percentile graph about a female. FIGS. 5G and 5H illustrate a case in which cutoff=23.0 and 25.0, respectively, as graphs showing a percentile graph about a male. Referring to FIGS. 5A to 5H, similar to the first item 240 of FIG. 2, distinguishing areas from the graph may be displayed together. For example, in FIGS. 5A to 5H, areas indicated with grids may correspond to “over,” areas indicated with oblique lines may correspond to “normal,” and areas indicated with dots may correspond to “under.”

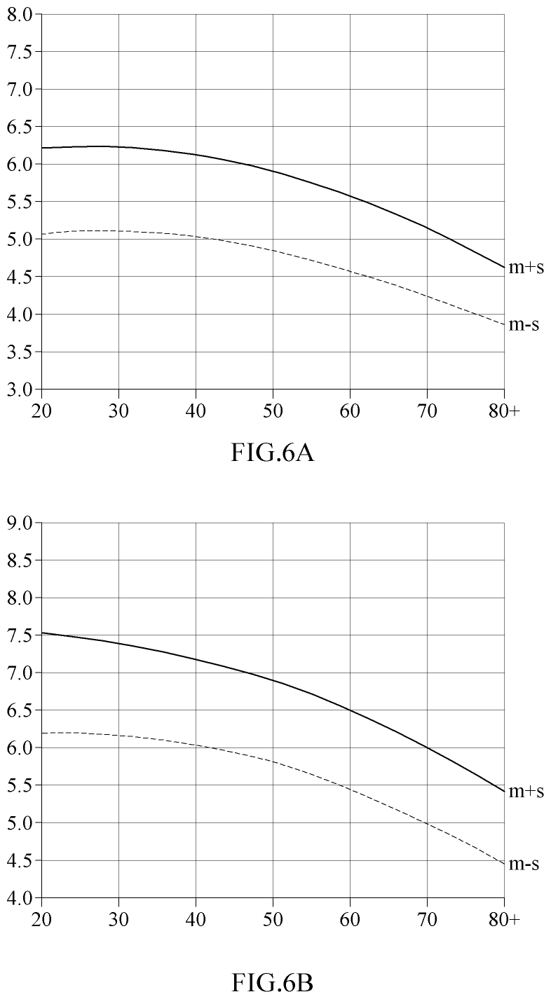

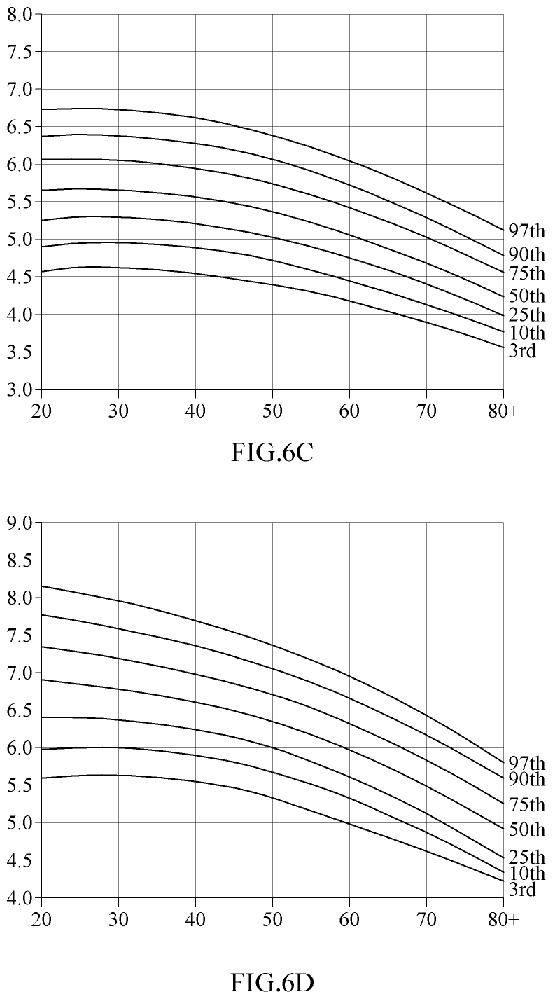

FIGS. 6A to 6D illustrate graphs corresponding to Whole Body Phase Angle_50 kHz (PhA, °). For example, FIG. 6A illustrates a graph showing the upper limit and the lower limit of the standard range about a female and FIG. 6B illustrates a graph showing the upper limit and the lower limit of the standard range about a male. Also, FIG. 6C illustrates a percentile graph about a female and FIG. 6D illustrates a percentile graph about a male.

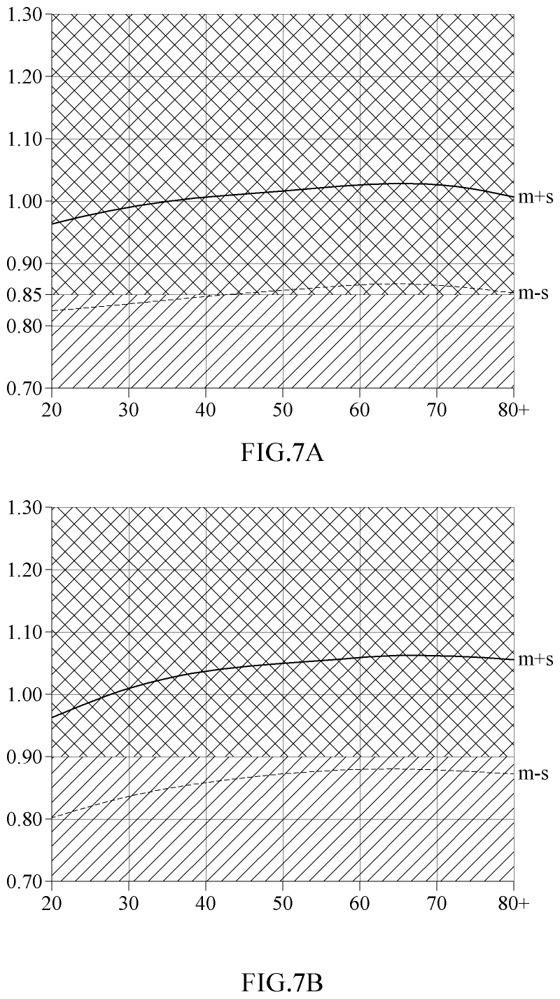

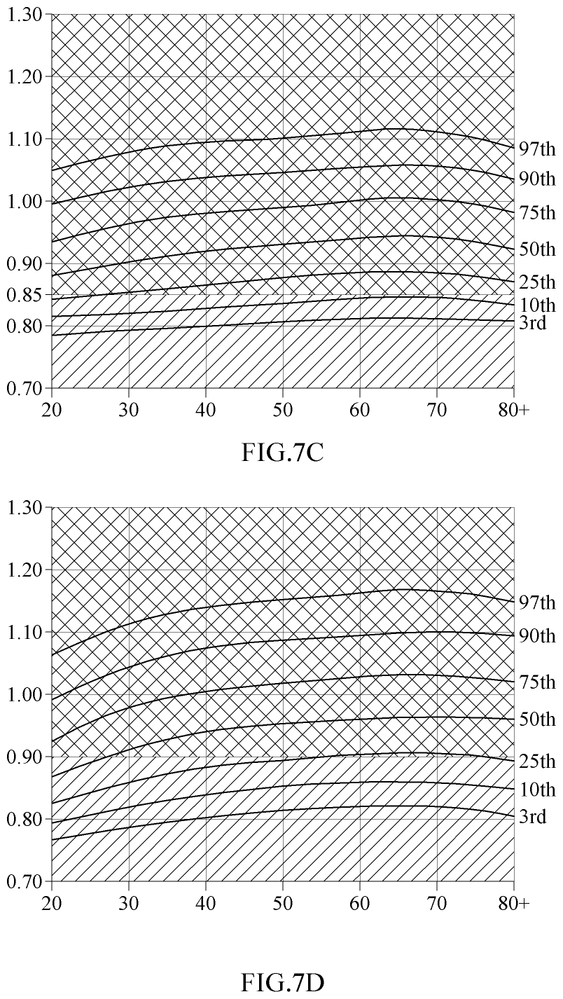

FIGS. 7A to 7D illustrate graphs corresponding to a Waist Hip Ratio (WHR). For example, FIG. 7A illustrates a graph showing the upper limit and the lower limit of the standard range about a female and FIG. 7B illustrates a graph showing the upper limit and the lower limit of the standard range about a male. Also, FIG. 7C illustrates a percentile graph about a female and FIG. 7D illustrates a percentile graph about a male. Referring to FIGS. 7A to 7D, similar to the first item 240 of FIG. 2, distinguishing areas from the graph may be displayed together. For example, in FIGS. 7A to 7D, areas indicated with grids may correspond to “over” and areas indicated with oblique lines may correspond to “normal.”

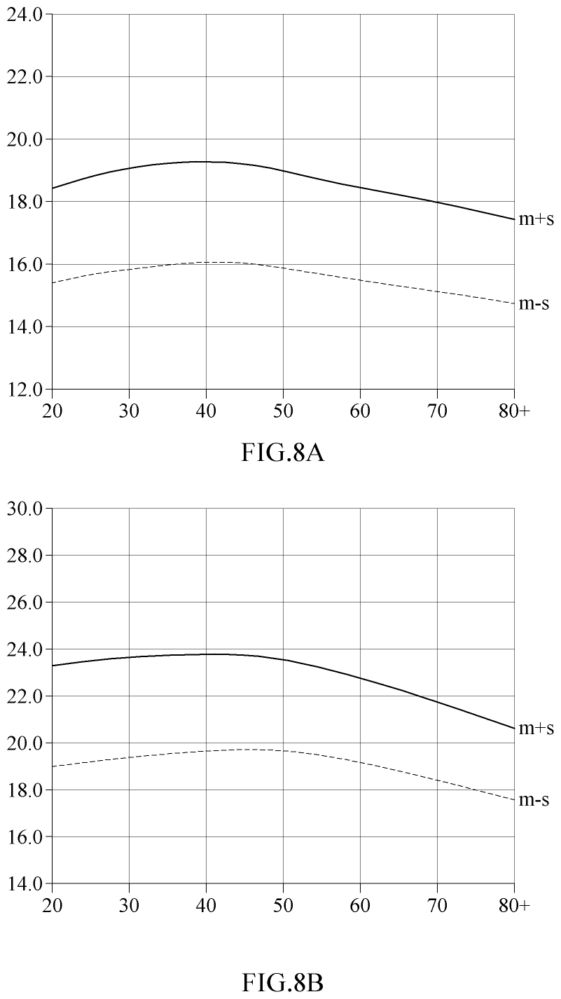

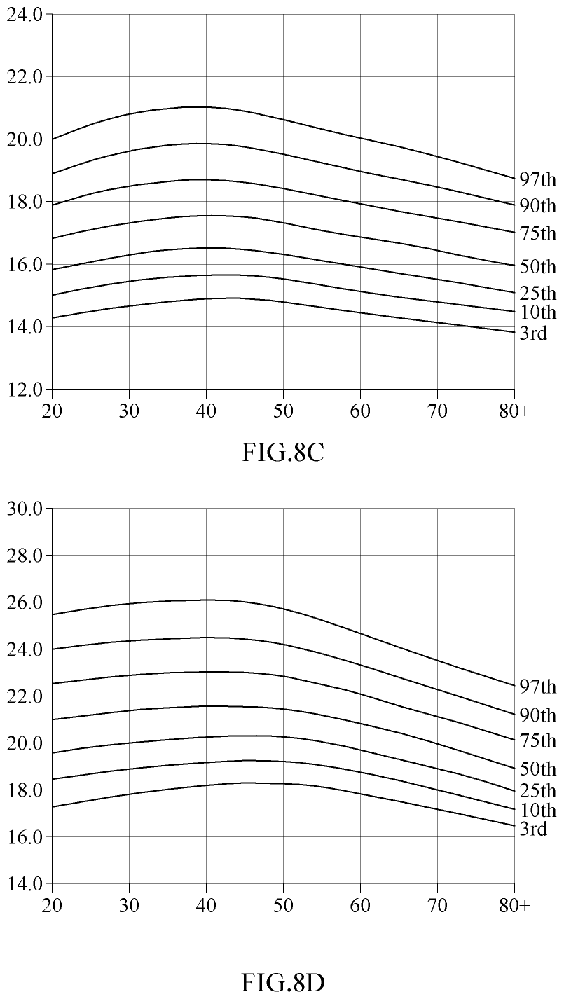

FIGS. 8A to 8D illustrate graphs corresponding to a Fat Free Mass Index (FFMI, kg/m²). For example, FIG. 8A illustrates a graph showing the upper limit and the lower limit of the standard range about a female and FIG. 8B illustrates a graph showing the upper limit and the lower limit of the standard range about a male. Also, FIG. 8C illustrates a percentile graph about a female and FIG. 8D illustrates a percentile graph about a male.

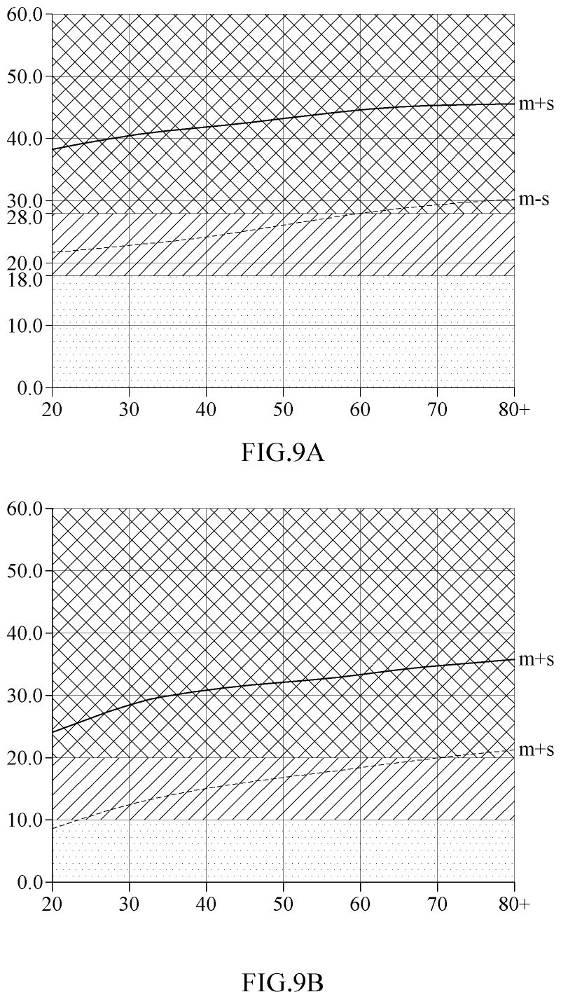

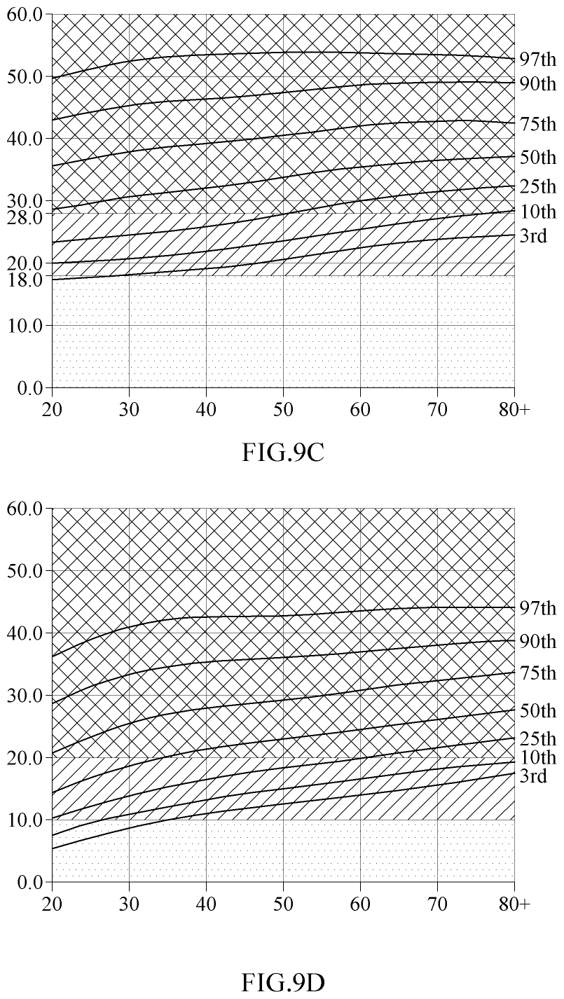

FIGS. 9A to 9D illustrate graphs corresponding to a Percent Body Fat (PBF, %). For example, FIG. 9A illustrates a graph showing the upper limit and the lower limit of the standard range about a female and FIG. 9B illustrates a graph showing the upper limit and the lower limit of the standard range about a male. Also, FIG. 9C illustrates a percentile graph about a female and FIG. 9D illustrates a percentile graph about a male. Referring to FIGS. 9A to 9D, similar to the first item 240 of FIG. 2, distinguishing areas from the graph may be displayed together. For example, in FIGS. 9A to 9D, areas indicated with grids may correspond to “over,” areas indicated with oblique lines may correspond to “normal,” and areas indicated with dots may correspond to “under.”

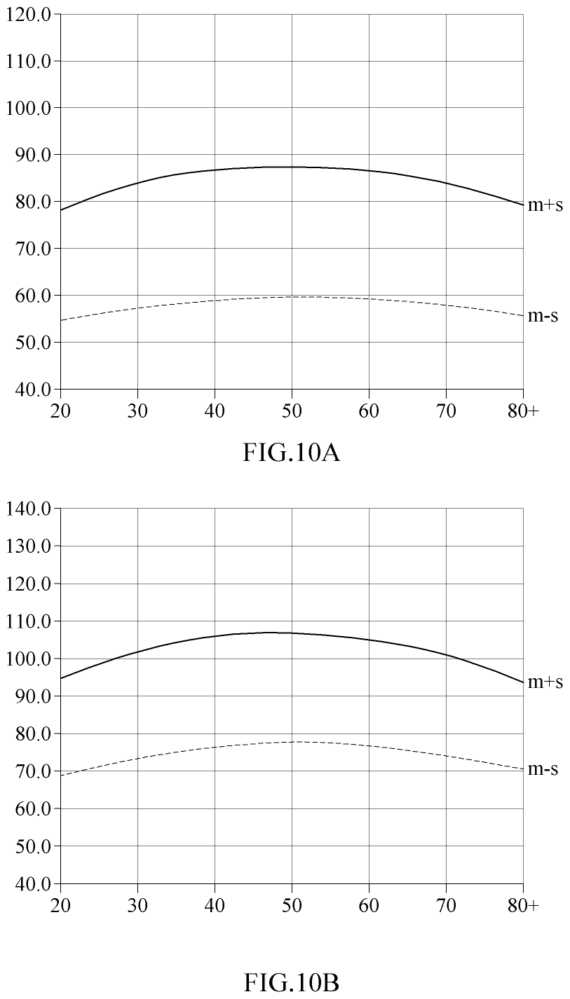

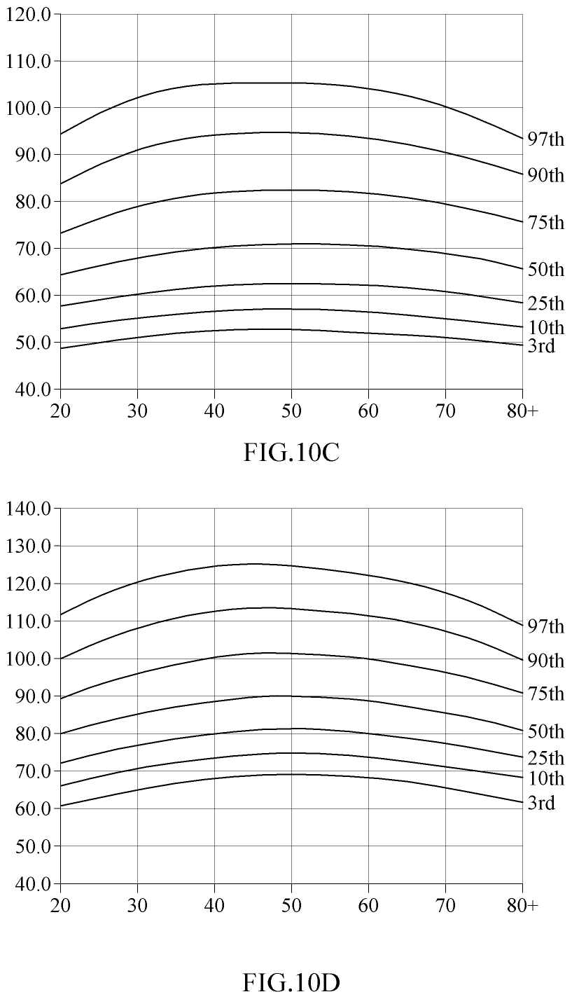

FIGS. 10A to 10D illustrate graphs corresponding to Weight (kg). For example, FIG. 10A illustrates a graph showing the upper limit and the lower limit of the standard range about a female and FIG. 10B illustrates a graph showing the upper limit and the lower limit of the standard range about a male. Also, FIG. 10C illustrates a percentile graph about a female and FIG. 10D illustrates a percentile graph about a male.

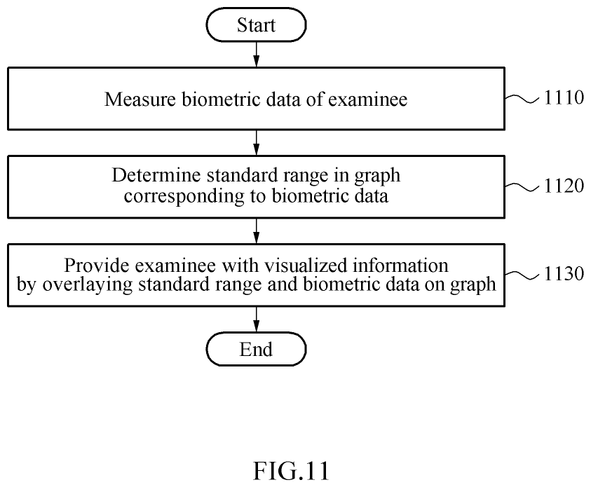

FIG. 11 is a flowchart illustrating a biometric data processing method according to an example embodiment.

The biometric data processing method according to an example embodiment may be performed by a processor included in a biometric data processing apparatus.

In operation 1110, the biometric data processing apparatus measures biometric data. In operation 1120, the biometric data processing apparatus determines a standard range in a graph corresponding to the biometric data. In operation 1130, the biometric data processing apparatus provides the examinee with visualized information by overlaying the standard range and the biometric data on the graph.

The description made above with reference to FIGS. 1 to 10D may apply alike to each of operations of FIG. 11 and thus, further description is omitted.

The example embodiments described above may be implemented using hardware components, software components, and/or a combination thereof. For example, the apparatuses, the methods, and the components described herein may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The software may include a computer program, a piece of code, an instruction, or some combination thereof, for independently or collectively instructing or configuring the processing device to operate as desired. Software and/or data may be embodied permanently or temporarily in any type of machine, component, physical equipment, virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more computer readable storage mediums.

The methods according to the above-described example embodiments may be recorded in computer-readable media in a form of a program instruction executable through various computer devices. The media may also include, alone or in combination with the program instructions, data files, data structures, and the like. The program instruction recorded in the media may be specially designed and configured for the example embodiments, or may be known to those in the field of computer software arts and thereby used. Examples of the media include magnetic media such as hard disks, floppy disks, and magnetic tapes; optical media such as CD-ROM discs and DVDs; magneto-optical media such as floptical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be to act as one or more software modules in order to perform the operations of the above-described example embodiments, or vice versa.

The components described herein may be implemented by hardware components including at least one of at least one digital signal processor (DSP), processor, controller, application specific integrated circuit (ASIC), programmable logic element such as field programmable gate array (FPGA), other electronic devices, and combination thereof. At least a portion of functions or processes described herein may be implemented by software. The corresponding software may be recorded in a recording medium. The components, functions, and processes described herein may be implemented through combination of hardware and software.

While the example embodiments are described with reference to the limited drawings, it will be apparent to one of ordinary skill in the art that various alterations and modifications in form and details may be made in these example embodiments. For example, suitable results may be achieved if the described techniques are performed in different order, and/or if components in a described system, architecture, device, or circuit are combined in a different manner, and/or replaced or supplemented by other components or their equivalents.

What is claimed is:

1. A biometric data processing method comprising:
measuring biometric data of an examinee;
determining a standard range in a graph corresponding to the biometric data; and
providing the examinee with visualized information by overlaying the standard range and the biometric data on the graph,
wherein the graph comprises one axis based on age and another axis based on the biometric data.

2. The biometric data processing method of claim 1, wherein
the biometric data comprises Whole Body ECW Ratio (ECW/TBW) data about a female,
the standard range is a range that is determined according to the age based on pre-stored reference data,
at the age of 20, the upper limit of the standard range has a value of 0.382 or more and 0.383 or less, and the lower limit of the standard range has a value of 0.370 or more and 0.372 or less,
at the age of 30, the upper limit of the standard range has a value of 0.383 or more and 0.384 or less, and the lower limit of the standard range has a value of 0.372 or more and 0.373 or less,
at the age of 40, the upper limit of the standard range has a value of 0.384 or more and 0.386 or less, and the lower limit of the standard range has a value of 0.373 or more and 0.375 or less,
at the age of 50, the upper limit of the standard range has a value of 0.386 or more and 0.389 or less, the lower limit of the standard range has a value of 0.375 or more and 0.378 or less, and
at the age of 60, the upper limit of the standard range has a value of 0.390 or more and 0.394 or less, and the lower limit of the standard range has a value of 0.379 or more and 0.382 or less.

3. The biometric data processing method of claim 1, wherein
the biometric data comprises Visceral Fat Area (VFA) data about a female,
the standard range is a range that is determined according to the age based on pre-stored reference data,
at the age of 20, the upper limit of the standard range has a value of 112.0 or more and 124.3 or less, and the lower limit of the standard range has a value of 39.3 or more and 42.5 or less,
at the age of 30, the upper limit of the standard range has a value of 120.9 or more and 147.7 or less, and the lower limit of the standard range has a value of 42.8 or more and 50.0 or less,
at the age of 40, the upper limit of the standard range has a value of 142.8 or more and 159.7 or less, and the lower limit of the standard range has a value of 49.8 or more and 58.4 or less,
at the age of 50, the upper limit of the standard range has a value of 154.8 or more and 175.4 or less, and the lower limit of the standard range has a value of 58.3 or more and 69.2 or less, and
at the age of 60, the upper limit of the standard range has a value of 172.3 or more and 186.7 or less, and the lower limit of the standard range has a value of 66.9 or more and 80.4 or less.

4. The biometric data processing method of claim 1, wherein
the biometric data comprises Body Mass Index (BMI) data about a female,
the standard range is a range that is determined according to the age based on pre-stored reference data,
at the age of 20, the upper limit of the standard range has a value of 28.9 or more and 30.9 or less, and the lower limit of the standard range has a value of 20.7 or more and 21.4 or less,
at the age of 30, the upper limit of the standard range has a value of 31.0 or more and 31.9 or less, and the lower limit of the standard range has a value of 21.5 or more and 22.1 or less,
at the age of 40, the upper limit of the standard range has a value of 32.0 or more and 32.5 or less, and the lower limit of the standard range has a value of 22.2 or more and 22.6 or less,
at the age of 50, the upper limit of the standard range has a value of 32.2 or more and 32.4 or less, and the lower limit of the standard range has a value of 22.4 or more and 22.6 or less, and
at the age of 60, the upper limit of the standard range has a value of 32.0 or more and 32.5 or less, and the lower limit of the standard range has a value of 22.3 or more and 22.5 or less.

5. The biometric data processing method of claim 1, wherein
the biometric data comprises Whole Body Phase Angle data about a female,
the standard range is a range that is determined according to the age based on pre-stored reference data,
at the age of 20, the upper limit of the standard range has a value of 6.20 or more and 6.28 or less, and the lower limit of the standard range has a value of 5.09 or more and 5.13 or less,
at the age of 30, the upper limit of the standard range has a value of 6.15 or more and 6.21 or less, and the lower limit of the standard range has a value of 5.05 or more and 5.11 or less,
at the age of 40, the upper limit of the standard range has a value of 5.94 or more and 6.15 or less, and the lower limit of the standard range has a value of 4.88 or more and 5.08 or less,
at the age of 50, the upper limit of the standard range has a value of 5.63 or more and 5.93 or less, and the lower limit of the standard range has a value of 4.60 or more and 4.86 or less, and
at the age of 60, the upper limit of the standard range has a value of 5.19 or more and 5.58 or less, and the lower limit of the standard range has a value of 4.28 or more and 4.57 or less.

6. The biometric data processing method of claim 1, wherein
the biometric data comprises Waist Hip Ratio (WHR) data about a female,
the standard range is a range that is determined according to the age based on pre-stored reference data,
at the age of 20, the upper limit of the standard range has a value of 0.97 or more and 0.99 or less, and the lower limit of the standard range has a value of 0.83 or more and 0.84 or less,
at the age of 30, the upper limit of the standard range has a value of 0.99 or more and 1.01 or less, and the lower limit of the standard range has a value of 0.84 or more and 0.85 or less,
at the age of 40, the upper limit of the standard range has a value of 1.01 or more and 1.02 or less, and the lower limit of the standard range has a value of 0.85 or more and 0.86 or less,
at the age of 50, the upper limit of the standard range has a value of 1.02 or more and 1.03 or less, and the lower limit of the standard range has a value of 0.86 or more and 0.87 or less, and
at the age of 60, the upper limit of the standard range has a value of 1.03 or more and 1.03 or less, and the lower limit of the standard range has a value of 0.87 or more and 0.87 or less.

7. The biometric data processing method of claim 1, wherein
the biometric data comprises Fat Free Mass Index (FFMI) data about a female,
the standard range is a range that is determined according to the age based on pre-stored reference data,
at the age of 20, the upper limit of the standard range has a value of 18.5 or more and 19.1 or less, and the lower limit of the standard range has a value of 15.5 or more and 15.8 or less,
at the age of 30, the upper limit of the standard range has a value of 19.1 or more and 19.3 or less, and the lower limit of the standard range has a value of 15.8 or more and 16.1 or less,
at the age of 40, the upper limit of the standard range has a value of 19.1 or more and 19.3 or less, and the lower limit of the standard range has a value of 16.0 or more and 16.1 or less,
at the age of 50, the upper limit of the standard range has a value of 18.5 or more and 19.0 or less, and the lower limit of the standard range has a value of 15.6 or more and 15.9 or less, and
at the age of 60, the upper limit of the standard range has a value of 18.1 or more and 18.4 or less, and the lower limit of the standard range has a value of 15.2 or more and 15.5 or less.

8. The biometric data processing method of claim 1, wherein
the biometric data comprises Percent Body Fat (PBF) data,
the standard range is a range that is determined according to the age based on pre-stored reference data,
at the age of 20, the upper limit of the standard range has a value of 38.6 or more and 40.8 or less, and the lower limit of the standard range has a value of 22.0 or more and 23.0 or less,
at the age of 30, the upper limit of the standard range has a value of 40.9 or more and 42.1 or less, and the lower limit of the standard range has a value of 23.3 or more and 24.4 or less,
at the age of 40, the upper limit of the standard range has a value of 42.1 or more and 43.5 or less, and the lower limit of the standard range has a value of 24.6 or more and 26.3 or less,
at the age of 50, the upper limit of the standard range has a value of 43.5 or more and 44.7 or less, and the lower limit of the standard range has a value of 26.3 or more and 28.2 or less, and
at the age of 60, the upper limit of the standard range has a value of 44.9 or more and 45.8 or less, and the lower limit of the standard range has a value of 28.1 or more and 29.7 or less.

9. The biometric data processing method of claim 1, wherein
the biometric data comprises Weight data about a female,
the standard range is a range that is determined according to the age based on pre-stored reference data,
at the age of 20, the upper limit of the standard range has a value of 78.6 or more and 84.1 or less, and the lower limit of the standard range has a value of 55.2 or more and 57.3 or less,
at the age of 30, the upper limit of the standard range has a value of 84.3 or more and 86.8 or less, and the lower limit of the standard range has a value of 57.3 or more and 59.1 or less,
at the age of 40, the upper limit of the standard range has a value of 86.9 or more and 88.0 or less, and the lower limit of the standard range has a value of 59.2 or more and 60.1 or less,
at the age of 50, the upper limit of the standard range has a value of 86.7 or more and 88.0 or less, and the lower limit of the standard range has a value of 59.5 or more and 60.1 or less, and
at the age of 60, the upper limit of the standard range has a value of 84.5 or more and 86.8 or less, and the lower limit of the standard range has a value of 58.2 or more and 59.3 or less.
